(19) United States
(12) Patent Application Publication
(10) Pub. No.: US 2016/0317111 A1
(43) Pub. Date: Nov. 3, 2016

(54) X-RAY REAL-TIME IMAGING DEVICE

(71) Applicant: BEIJING EAST WHALE IMAGE TECHNOLOGY CO., LTD., Beijing (CN)

(72) Inventors: Xun ZHU, Yizhuang, Beijing (CN); Xingbai HE, Yizhuang, Beijing (CN); Jun ZHANG, Yizhuang, Beijing (CN)

(21) Appl. No.: 15/105,804

(22) PCT Filed: Jan. 17, 2014
(86) PCT No.: PCT/CN2014/000058

§ 371 Date: Jun. 17, 2016

(51) Int. Cl. A61B 6/00

(52) U.S. Cl. CPC A61B 6/4441; A61B 6/4014; A61B 6/4452; A61B 6/4464; A61B 6/4482

(57) ABSTRACT

An X-ray real-time imaging device includes a first C-shaped arm slidably disposed on a second C-shaped arm, wherein the first C-shaped arm is provided with a first X-ray generating device and a first video receiving device, thereby constituting a first imaging device, and the second C-shaped arm is provided with a second X-ray generating device and a second video receiving device, thereby constituting a second imaging device, wherein the axis of rotation of the first C-shaped arm is the same as that of the second C-shaped arm. When the first C-shaped arm slides along the second C-shaped arm, different angles can form between the first and second imaging devices, and as the axes of rotation of the first and second arms are the same, the axis of rotation of the first imaging device is the same as the axis of rotation of the second imaging device.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

This invention relates to an X-ray imaging device, especially an X-ray real-time imaging device.

2. Description of Related Art Including Information Disclosed Under 37 CFR 1.97 and 37 CFR 1.98.

As the biplane imaging device has many advantages, for example, it can position patients more accurately, help doctors shorten the operation duration, reduce the exposure dose, keep a sterile environment, etc., it is widely used in operations.

The biplane imaging device can be realized with the device in U.S. Pat. No. 4,884,293. U.S. Pat. No. 4,884,293 describes a biplane imaging system. One imaging system is fixed on the floor of the operating room and the other imaging system is hung on the guide rail of the ceiling. The device has a disadvantage that accurate match is required upon the initial installation of the two systems; otherwise the axes of rotation of the two systems will be different easily.

The said biplane imaging device can also be realized with the device in U.S. Pat. No. 3,549,885. U.S. Pat. No. 3,549,885 describes a biplane imaging system. The two systems are installed in a rotatable ring in a mutually-perpendicular manner. The device has a disadvantage that the angle between two systems is always 90 degrees and can't be changed.

The said biplane imaging system can also be realized with the device in U.S. Pat. No. 5,095,501. U.S. Pat. No. 5,095,501 describes two imaging systems which are installed in a rotatable ¾ ring in a mutually-perpendicular manner to form a rotatable G-shaped arm. The device has a disadvantage that the angle between the two imaging systems is always 90 degree and can't be changed.

### BRIEF SUMMARY OF THE INVENTION

This invention aims to provide an X-ray real-time imaging device where the two systems share the same axis of rotation and the angle between them is variable.

This invention adopts the following technical schemes to solve technical problems: an X-ray real-time imaging device including a first C-shaped arm, a second C-shaped arm and a supporting device. The first C-shaped arm is slidably disposed on the second C-shaped arm and the second C-shaped arm is slidably disposed on the supporting device. The two ends of the first C-shaped arm along the circumference are respectively provided with a first X-ray generating device and a first video receiving device. The two ends of the second C-shaped arm along the circumference are respectively provided with a second X-ray generating device and a second video receiving device. The intersecting line of the X-ray generating plane of the first X-ray generating device and the X-ray generating plane of the second X-ray generating device is the axis of rotation. The first X-ray generating device and the first video receiving device slide along the axis of rotation driven by the first C-shaped arm, and the second X-ray generating device and the second video receiving device also slide along the axis of rotation driven by the second C-shaped arm.

Alternatively, the second C-shaped arm contains a curved guide rail with a curved sliding block. The first C-shaped arm is disposed on the curved sliding block. The second C-shaped arm is provided with a first motor which drives the first C-shaped arm and the curved sliding block to slide along the curved guide rail.

Alternatively, the first C-shaped arm is provided with gear teeth in its inner side; the output axis of the first motor is provided with gears; the gears and the gear teeth are engaged.

Alternatively, the supporting device includes a C arm guide rail and a second motor. The second C-shaped arm is slidably disposed on the C arm guide rail. The second motor drives the second C-shaped arm to slide on the C arm guide rail.

Alternatively, the supporting device also includes a horizontal sliding arm. The C arm sliding rail can be rotatably disposed on the horizontal sliding arm.

Alternatively, the supporting device also includes a mobile car. The C arm guide rail can move straightly and horizontally in relation to the horizontal sliding arm. The horizontal sliding arm can be rotatably disposed on the mobile car and move vertically in relation to the mobile car.

Alternatively, the supporting device also includes a first base. The C arm guide rail can move straightly and horizontally in relation to the horizontal sliding arm. The horizontal sliding arm can be rotatably disposed on the first base and move vertically in relation to the first base.

Alternatively, the supporting device also includes a second base. The horizontal sliding arm can be rotatably disposed on the second base and move vertically in relation to the second base. The second base can be slidably disposed on the ceiling.

Alternatively, the X-ray real-time imaging device also includes a control device. The imaging control device includes a first indicator, a second indicator and a controller. The first indicator and the second indicator are respectively connected to the signal of the controller. The controller is also connected to the signal of the first video receiving device and the second video receiving device.

Alternatively, the imaging control device also includes a foot switch. The signal of the foot switch is connected to the controller, and the signal of the controller is connected to the first X-ray generating device and the second X-ray generating device.

This invention has the following advantageous effects: The X-ray real-time imaging device includes a second C-shaped arm and a first C-shaped arm which can be slidably disposed on the second C-shaped arm; the two ends of the first C-shaped arm are provided with a first X-ray generating device and a first video receiving device; the first X-ray generating device and the first video receiving device constitute a first imaging device; the two ends of the second C-shaped arm are provided with a second X-ray generating device and a second video receiving device; the second X-ray generating device and the second video receiving device constitute a second imaging device. As the first C-shaped arm slides along the second C-shaped arm, the angle between the first imaging device and the second imaging device varies to form images of the detected objects from multiple directions. As the first C-shaped arm and the second C-shaped arm share the same axis of rotation, the first imaging device and the second imaging device share the same axis of rotation.

### BRIEF DESCRIPTION OF THE DRAWINGS

FIG. 1 is the schematic diagram of the structure of the X-ray real-time imaging device;

FIG. 2 is the schematic diagram of various operating conditions of the X-ray real-time imaging device;

FIG. 3 is the schematic diagram of the cross-section of the position relation between the first C-shaped arm and the second C-shaped arm;

FIG. 4 is another schematic diagram of the structure of the X-ray real-time imaging device;

FIG. 5 is another schematic diagram of the structure of the X-ray real-time imaging device;

FIG. 6 is the schematic diagram of the structure of the imaging control device of the X-ray real-time imaging device;

Marks in the figures: 1—first C-shaped arm; 2—second C-shaped arm; 4—first video receiving device; 6—second video receiving device; 7—curved guide rail; 8—curved sliding block; 9—first motor; 10—C arm guide rail; 11—second motor; 12—horizontal sliding arm; 13—mobile car; 14—first base; 15—second base; 16—first indicator; 17—second indicator; 18—controller; 19—foot switch; 20—first X-ray generating device; 21—first beam limiter; 22—second X-ray generating device; 23—second beam limiter.

### DETAILED DESCRIPTION OF THE INVENTION

This part further explains the attached figures and the technical scheme of this invention in combination with embodiments.

### Embodiment 1

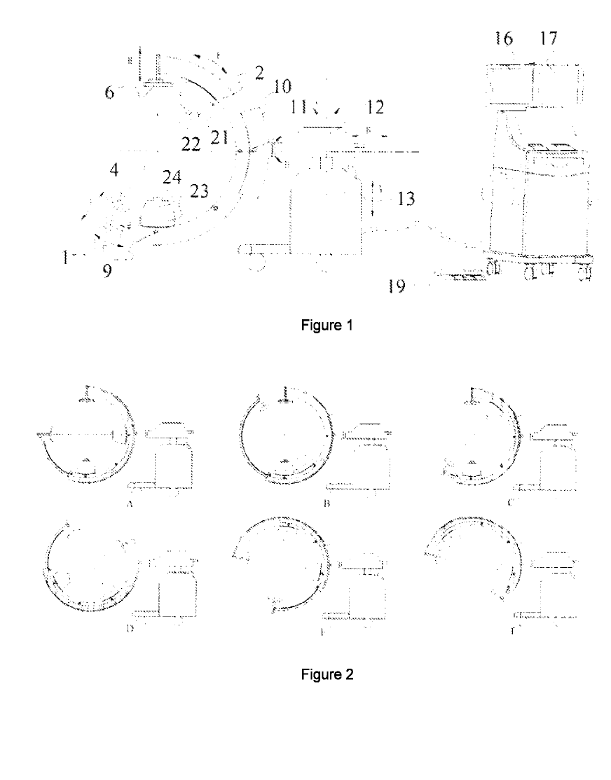

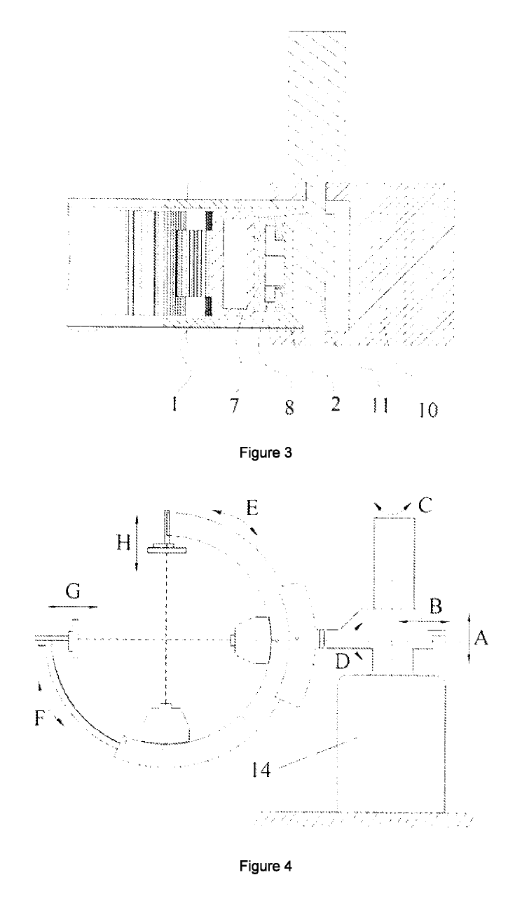

See FIGS. 1-6. This embodiment provides an X-ray real-time imaging device including a first C-shaped arm, a second C-shaped arm 2 and a supporting device. The first C-shaped arm is slidably disposed on the second C-shaped arm. See the direction F in FIG. 1. The second C-shaped arm is slidably disposed on the supporting device. The two ends of the first C-shaped arm along the circumference are respectively provided with a first X-ray generating device and a first video receiving device 4. The two ends of the second C-shaped arm along the circumference are respectively provided with a second X-ray generating device and a second video receiving device 6. The intersecting line of the X-ray generating plane of the first X-ray generating device and the X-ray generating plane of the second X-ray generating device is the axis of rotation. The first X-ray generating device and the first video receiving device slide along the axis of rotation driven by the first C-shaped arm, and the second X-ray generating device and the second video receiving device also slide along the axis of rotation driven by the second C-shaped arm, which means the axis of rotation of the first C-shaped arm 1 is the same as the axis of rotation of the second C-shaped arm 2. The X-ray real-time imaging device of this invention includes a second C-shaped arm and a first C-shaped arm 1 which can be slidably disposed on the second C-shaped arm. The two ends of the first C-shaped arm 1 are provided with a first X-ray generating device and a first video receiving device 4. The first X-ray generating device and the first video receiving device 4 constitute a first imaging device. The two ends of the second C-shaped arm 1 are provided with a second X-ray generating device and a second video receiving device 6. The second X-ray generating device and the second video receiving device 6 constitute a second imaging device. As the first C-shaped arm 1 slides along the second C-shaped arm 2, the angle between the first imaging device and the second imaging device varies to form images of the detected object from multiple directions. As the first C-shaped arm and the second C-shaped arm share the same axis of rotation, the first imaging device and the second imaging device share the same axis of rotation.

In this embodiment, alternatively, the first video receiving device 4 is slidably disposed on one end of the first C-shaped arm 1 along the connecting line between the first X-ray generating device and the first video receiving device 4, which means it can make translational motion along the direction G shown in FIG. 1, and the second video receiving device 6 is slidably disposed on one end of the second C-shaped arm 2 along the connecting line between the second X-ray generating device and the second video receiving device 6, which means it can make translational motion along the direction H shown in FIG. 1, to adjust the distance between the first X-ray generating device and the first video receiving device 4 and the distance between the second X-ray generating device and the second video receiving device 6, so as to enhance the definition of the image generated by the first imaging device and the second imaging device.

In this embodiment, alternatively, the second C-shaped arm 2 contains a curved guide rail 7 on which a curved block 8 is disposed. The first C-shaped arm 1 is disposed on the curved block 8. The second C-shaped arm 2 is provided with a first motor 9. The first motor 9 drives the first C-shaped arm 1 and the curved block 8 to slide along the curved guide rail 7, to enhance the accuracy of the first C-shaped arm's sliding along the second C-shaped arm 2 through the coordination between the curved guide rail 7 and the curved sliding block 8, so that axis of rotation 0 is not affected by the sliding of the first C-shaped arm, thus enhancing the quality of images generated by the first imaging device and the second imaging device.

In this embodiment, alternatively, the first C-shaped arm 1 is provided with gear teeth in its inner side; the output axis of the first motor 9 is provided with gears; the gears and the gear teeth are engaged to allow the first motor 9 to drive the first C-shaped arm 1. Preferably, the first motor 9 acts as a DC servo motor or an AC servo motor to enhance the motion control accuracy of the first motor 9 and enhance the motion accuracy of the first C-shaped arm 1.

In this embodiment, alternatively, the supporting device also includes C arm guide rail 10 and the second motor 11; the second C-shaped arm is slidably disposed on the C arm sliding rail 10; the second motor 11 drives the second C-shaped arm 2 to move along the C arm sliding rail 10, which allows the second C-shaped arm to move in the direction E shown in FIG. 1, thus ensuring the first C-shaped arm 1 and the second C-shaped arm 2 to easily pass through objects such as an operating table and expanding the application scope of the X-ray real-time imaging device. Besides, preferably, the second C-shaped arm 2 and the second motor 11 are disposed on the C arm sliding rail 10 in a belt bearing manner, i.e. the manner in the existing technology.

In this embodiment, alternatively, the supporting device also includes a horizontal sliding arm 12; the C arm sliding rail 10 can be rotatably disposed on the horizontal sliding arm 12, which means it can rotate along the direction D in FIG. 1, so that the X-ray real-time imaging device has multiple operable free degrees and doctors can control the end actuator of the X-ray real-time imaging device, i.e. the pose of the first C-shaped arm 1 and the second C-shaped arm 2. Besides, preferably, the C arm sliding rail 10 is disposed on the horizontal sliding arm 12 through the first driving mechanism to realize its rotation in relation to the horizontal sliding arm 12.

In this embodiment, alternatively, the supporting device also includes a mobile car 13; the C arm sliding rail 0 can make linear and horizontal motion along the horizontal sliding arm 12, which means the C arm sliding rail 10 can rotate along the direction D shown in FIG. 1 and the horizontal sliding arm 12 can be rotatably disposed on the mobile car 13. It means the horizontal sliding arm 12 can rotate along the direction C shown in FIG. 1 and make linear and vertical motion in relation to the mobile car 13, which means it can make translational motion along the direction A shown in FIG. 1, so that the X-ray real-time imaging device has multiple free degrees and doctors can control the end actuator of the X-ray real-time imaging device, i.e. the pose of the first C-shaped arm and the second C-shaped arm. Besides, preferably, the C arm sliding rail 10 is disposed on the horizontal sliding arm 12 through the first driving mechanism and the first sliding mechanism; the first driving mechanism is disposed on the first sliding mechanism to realize the rotation and translational motion of the C arm sliding rail 10 in relation to the horizontal sliding arm 12. The horizontal sliding arm 12 is disposed on the mobile car 13 through the second driving mechanism and the second sliding mechanism. The second driving mechanism is disposed on the second sliding mechanism to realize the rotation and translational motion of the horizontal sliding arm 12 in relation to the mobile car 13.

In this embodiment, alternatively, the supporting device includes a first base 14. The C arm sliding rail 10 can make linear and horizontal motion in relation to the horizontal arm 12, which means it can make translational motion along the direction B shown in FIG. 4. The horizontal sliding arm 12 can be rotatably disposed on the first base 14, which means it can rotate along the direction C shown in FIG. 4, make linear and vertical motion in relation to the first base 14, and make translational motion along the direction A shown in FIG. 4. The first base 14 is fixed on the ground, so that the X-ray real-time imaging device has multiple free degrees and doctors can easily control the end actuator of the X-ray real-time imaging device, i.e. the pose of the first C-shaped arm 1 and the second C-shaped arm 2. In addition, by fixing the first base 14 on the ground, the coordination accuracy between the X-ray real-time imaging device and the operating table can be enhanced and the quality of operation can be further improved. Besides, preferably, the C arm sliding rail 10 is disposed on the horizontal sliding arm 12 through the first driving mechanism and the third sliding mechanism, and the first driving mechanism is disposed on the third sliding mechanism to realize the rotation and translational motion of the C arm sliding rail 10 in relation to the horizontal sliding arm 12. The horizontal sliding arm 12 is disposed on the first base 14 through the third driving mechanism and the fourth sliding mechanism, and the third driving mechanism is disposed on the fourth sliding mechanism to realize the rotation and translational motion of the horizontal sliding arm 12 in relation to the first base 14.

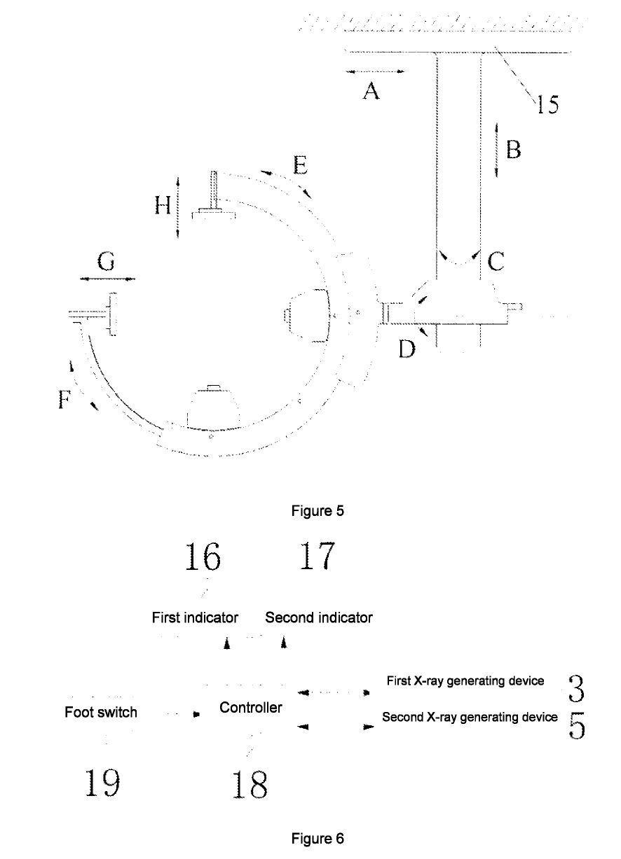

In this embodiment, alternatively, the supporting device also includes a second base 15. The horizontal sliding arm 14 is rotatably disposed on the second base 15, which means it can rotate along the direction C shown in FIG. 5, make linear and vertical motion in relation to the second base 15 and make translational motion along the direction B shown in FIG. 5. The second base 15 can be slidably disposed on the ceiling, which means it can make translational motion along the direction A shown in FIG. 5, so that the X-ray real-time imaging device has multiple free degrees and doctors can easily control the end actuator of the X-ray real-time imaging device, i.e. the pose of the first C-shaped arm 1 and the second C-shaped arm 2. In addition, by fixing the second base 15 on the ceiling, the sterile environment of the operating room can be better protected. Preferably, the ceiling is provided with a guide rail, and the second base 15 is slidably disposed on the guide rail to realize the high-precision motion of the second base 15 along the guide rail.

In this embodiment, alternatively, an imaging control device is included. The imaging control device includes a first indicator 16, a second indicator 17 and a controller 18. The first indicator 16 and the second indicator 17 are respectively connected to the signal of the controller 18. The controller 18 is connected to the signals of the first video receiving device 4 and the second video receiving device 6 to display the images received by the first video receiving device 4 and the second video receiving device 6 on the first indicator 16 and the second indicator 17 respectively and realize the real-time imaging of the first imaging device and the second imaging device of the X-ray real-time imaging device.

In this embodiment, alternatively, the imaging control device also includes a foot switch 19. The signal of the foot switch 19 is connected to the controller 18 and the controller 18 connects the signal to the first X-ray generating device and the second X-ray generating device, so as to control whether the first X-ray generating device and/or the second X-ray generating device participate(s) in the imaging of the X-ray real-time imaging device.

In this embodiment, alternatively, the first X-ray generating device includes a first X-ray generator 20 and a first bean limiter 21, and/or the second X-ray generating device includes a second X-ray generator 22 and a second bean limiter 23, to block the unnecessary primary X-ray with the beam limiter and control the radiation field of X-ray within the minimum range required, thus minimizing the dose of X-ray received by patients.

The order of embodiments above is for the explanatory purpose only and doesn't represent the advantages and disadvantages of the embodiments.

Last, it should be noted: The above embodiments are only used to explain the technical scheme of this invention instead of limiting the scheme. Although detailed explanations are made to this invention with reference to the above embodiments, technical personnel in this field shall understand that they can modify the technical scheme recording the above embodiments or replace part of technical characteristics therein. These modifications and replacements shall not cause the essence of the corresponding technical scheme to deviate from the spirit and scope of the technical schemes of embodiments of this invention.

1. An X-ray real-time imaging device with the following characteristics: It includes a first C-shaped arm, a second C-shaped arm and a supporting device. The first C-shaped arm is slidably disposed on the second C-shaped arm; the second C-shaped arm is slidably disposed on the supporting device; the two ends of the first C-shaped arm along the circumference are respectively provided with a first X-ray generating device and a first video receiving device; the two ends of the second C-shaped arm along the circumference are respectively provided with a second X-ray generating device and a second video receiving device; the intersecting line of the X-ray generating plane of the first X-ray generating device and the X-ray generating plane of the second X-ray generating device is the axis of rotation. The first X-ray generating device and the first video receiving device slide along the axis of rotation driven by the first C-shaped arm, and the second X-ray generating device and the second video receiving device also slide along the axis of rotation driven by the second C-shaped arm.

2. An X-ray real-time imaging device as claimed in claim 1, wherein the second C-shaped arm contains a curved guide rail; a curved sliding block is disposed on the curved guide rail; the first C-shaped arm is disposed on the curved sliding block; the second C-shaped arm is provided with a first motor which drives the first C-shaped arm and the curved sliding block to slide along the curved guide rail.

3. An X-ray real-time imaging device as claimed in claim 1, wherein the first C-shaped arm is provided with gear teeth in its inner side; the output axis of the first motor is provided with gears; the gears and the gear teeth are engaged.

4. An X-ray real-time imaging device as claimed in claim 1, wherein the supporting device includes a C arm sliding rail and a second motor; the second C-shaped arm is slidably disposed on the C arm sliding rail; the second motor drives the second C-shaped arm to slide along the C arm sliding rail.

5. An X-ray real-time imaging device as claimed in claim 4, wherein the supporting device also includes a horizontal sliding arm and the C arm sliding rail can be slidably disposed on the horizontal sliding arm.

6. An X-ray real-time imaging device as claimed in claim 5, wherein the supporting device also includes a mobile car; the C arm sliding rail can make linear and horizontal motion in relation to the horizontal sliding arm; the horizontal sliding arm can be rotatably disposed on the mobile car and make vertical motion in relation to the mobile car.

7. An X-ray real-time imaging device as claimed in claim 5, wherein the supporting device also includes a first base; the C arm sliding rail can make linear and horizontal motion in relation to the horizontal sliding arm; the horizontal sliding arm is rotatably disposed on the first base and can make vertical motion in relation to the first base; the first base is fixed on the ground.

8. An X-ray real-time imaging device as claimed in claim 5, wherein the supporting device also includes a second base; the horizontal sliding arm is rotatably disposed on the second base and can make vertical motion in relation to the second base; the second base is slidably disposed on the ceiling.

9. An X-ray real-time imaging device as claimed in claim 1, wherein an imaging control device is included. The imaging control device includes a first indicator, a second indicator and a controller. The first indicator and the second indicator are respectively connected to the signal of the controller; the controller is connected to the signals of the first video receiving device and the second video receiving device.

10. An X-ray real-time imaging device as claimed in claim 9, wherein the imaging control device also includes a foot switch. The signal of the foot switch is connected to the controller and the controller connects the signal to the first X-ray generating device and the second X-ray generating device.
